(19) United States
(12) Patent Application Publication
(10) Pub. No.: US 2026/0256415 A1
(43) Pub. Date: Sep. 3, 2026

(54) System and Method for Detecting a Seizure in a Subject

(71) Applicant: Amrita Vishwa Vidyapeetham, Coimbatore (IN)

(72) Inventors: C. Santhosh Kumar, Palakkad (IN); Pooja Muralidharan, Mumbai (IN); K. I. Ramachandran, Coimbatore (IN); Ravi Sankaran, Flint, MI (US)

(73) Assignee: Amrita Vishwa Vidyapeetham, Coimbatore (IN)

(21) Appl. No.: 19/282,801

(22) Filed: Jul. 28, 2025

(30) Foreign Application Priority Data
Feb. 28, 2025 (IN) 202541048107

(51) Int. Cl. A61B 5/00; A61B 5/024; A61B 5/16; A61B 5/364; G16H 50/20; G16H 50/30

(52) U.S. Cl. CPC A61B 5/4094; A61B 5/02405; A61B 5/165; A61B 5/364; A61B 5/4812; A61B 5/7203; A61B 5/746; G16H 50/20; G16H 50/30

(57) ABSTRACT

The present invention relates to a system and a method for detecting a seizure in a subject. The method includes receiving electrocardiogram (ECG) signals from sensors and identifying patterns associated with the ECG signals. Further, upon identification of the one or more patterns, the method includes extracting variations from the patterns. Further, the method includes predicting, an occurrence of the seizure in the subject within a predefined duration based on the extracted variations using Artificial Intelligence (AI) models. Further, the method includes transmitting an alert signal to at least one device upon prediction of the occurrence of the seizure in the subject. The system and the method enhance seizure detection accuracy by utilizing the AI models, ensuring real-time monitoring and enabling early seizure prediction for timely intervention.

### CROSS REFERENCE TO RELATED APPLICATIONS

The present application claims priority to an Indian Patent Application #202541048107 filed on Feb. 28, 2025. All disclosure of the parent application is incorporated at least by reference.

### BACKGROUND OF THE INVENTION

### Field of the Invention

The present disclosure generally relates to health monitoring systems, and more particularly relates to a system and method for detecting a seizure in a subject using electrocardiogram (ECG) signals, thereby enabling early seizure prediction and timely intervention before onset of the seizure.

### Description of Related Art

Epilepsy is a neurological disorder characterized by recurrent, unprovoked seizures. Seizures may vary in intensity and can happen unexpectedly, posing a substantial risk to patients. Predicting an occurrence of seizures before the onset of the seizures can significantly improve the quality of life for individuals with epilepsy, enabling them to take preventive actions.

Existing methods for detecting seizures during or after their occurrence largely rely on electroencephalogram (EEG) or invasive monitoring, which may not be suitable for continuous, real-time use or for everyday applications outside of clinical settings. Further, the existing methods often require specialized equipment and can be expensive and uncomfortable for patients.

Although some existing methods utilize other ECG based prediction models using small custom datasets or the models are trained and tested using data from the same patient in a patient-specific manner. The prediction models are not suitable for developing wearable devices. Further, the performance of ECG-based algorithms for patient-independent systems is not accurate enough for wearable devices. Existing solutions face challenges in accurately predicting seizures based on ECG signals due to the complexity of heart-brain interactions and the variety of seizure types.

Therefore, there is a need to address at least the above-mentioned drawbacks and any other shortcomings, or at the very least, provide a valuable alternative to the existing methods and systems.

### OBJECTS OF THE INVENTION

An object of the present disclosure relates to a system and method for detecting a seizure in a subject using electrocardiogram (ECG) signals, thereby enabling real-time seizure prediction.

Another object of the present disclosure provides a system for detecting a seizure by removing non-seizure related components from the ECG signals, thereby improving accuracy of seizure prediction.

Another object of the present disclosure is to provide a system for predicting seizures before onset of the seizure, thereby facilitating timely prediction of the seizure for taking preventive actions.

Another object of the present disclosure is to extract variations related only to the seizures, thereby ensuring precise identification of seizure-related variations and optimizing seizure prediction.

Yet another object of the present disclosure is to provide a score for detected seizure variations, thereby ensuring reduction of false positives during prediction.

### BRIEF SUMMARY OF THE INVENTION

Aspects of the present disclosure generally relate to health monitoring systems, and more particularly relates to a system and method for detecting a seizure in a subject using electrocardiogram (ECG) signals, thereby enabling early seizure prediction and timely intervention before onset of the seizure. Additionally, the system and the method utilize Artificial Intelligence (AI) models to extract and classify seizure-related characteristics from the ECG signals, allowing for proactive seizure management and potentially reducing the risk of seizure-related complications.

An aspect of the present disclosure relates to a method for detecting a seizure in a subject. The method may include receiving, by one or more processors associated with a system, one or more electrocardiogram (ECG) signals from one or more sensors associated with the system. The method may include identifying, by the one or more processors, one or more patterns associated with the one or more ECG signals and extracting, by the one or more processors, one or more variations from the one or more patterns, upon identification of the one or more patterns. The method may include predicting, by the one or more processors, an occurrence of the seizure in the subject within a predefined duration based on the extracted one or more variations using Artificial Intelligence (AI) models configured within the system. The method may include transmitting, by the one or more processors, an alert signal to at least one device upon prediction of the occurrence of the seizure in the subject.

In an embodiment, the one or patterns may include any one or a combination of: heart rate acceleration, cardiac rhythm, amplitude of the one or more ECG signals and frequency of the one or more ECG signals.

In an embodiment, the one or more variations may include any one or a combination of: heart rate variability, irregularities in the cardiac rhythm, changes in neural activity, cardiac cycle intervals, power spectral density of the frequency of the one or more ECG signals and waveform complexity.

In an embodiment, the method may include filtering, by the one or more processors, a noise from the received one or more ECG signals prior to identifying the one or more patterns.

In an embodiment, the method may include segmenting, by the one or more processors, the one or more ECG signals into predetermined time frames for identifying the one or more patterns.

In an embodiment, the extracting, by the one or more processors, the one or more variations from the one or more patterns, may include determining, by the one or more processors, a value of the one or more variations, comparing, by the one or more processors, the value with pre-stored value of the one or more variations in a database associated with the system, determining, by the one or more processors, that the value exceeds a predefined range based on the comparison and extracting, by the one or more processors, the one or more variations upon determination that the value exceeds the predefined range.

In an embodiment, the method may include receiving, by the one or more processors, one or more interictal ECG signals from the one or more sensors, determining, by the one or more processors, one or more parameters associated with the one or more interictal ECG signals and removing, by the one or more processors, the one or more parameters from the one or more patterns using one or more filtering techniques prior to extracting the one or more variations.

In an embodiment, the one or more parameters may include any one or a combination of: regular heartbeat patterns and non-seizure neural activity.

In an embodiment, the method may include determining, by the one or more processors, one or more attributes associated with the one or more ECG signals and removing, by the one or more processors, the one or more attributes from the one or more patterns prior to extracting the one or more variations.

In an embodiment, the one or more attributes may include any one or a combination of: patient stress levels, sleep status, and medication adherence.

In an embodiment, the method may include determining, by the one or more processors, one or more stages associated with the seizure based on the extracted one or more variations, classifying, by the one or more processors, the one or more variations into the one or more stages using the AI models upon extraction of the one or more variations to predict the occurrence of the seizure in the subject, assigning, by the one or more processors, a score to the one or more variations upon classification of the one or more variations into the one or more stages. The method may include determining, by the one or more processors, that the score exceeds a predefined threshold and detecting, by the one or more processors, the occurrence of the seizure in the subject within the predefined duration, upon determination that the score exceeds the predefined threshold.

In an embodiment, the one or more stages may include any of: preictal stage, ictal stage, interictal stage and postictal stage.

In an embodiment, the method may include validating, by the one or more processors, the occurrence of the seizure by comparing the predicted seizure with historical seizure data stored in the database.

Another embodiment of the present disclosure may include a system for detecting a seizure in a subject. The system may include one or more processors and a memory operatively coupled with the one or more processors. The memory may include one or more instructions which, when executed, cause the one or more processors to receive one or more electrocardiogram (ECG) signals from one or more sensors associated with the system. The processor may identify one or more patterns associated with the one or more ECG signals and extract one or more variations from the one or more patterns, upon identification of the one or more patterns. The processor may predict an occurrence of the seizure in the subject within a predefined duration based on the extracted one or more variations using Artificial Intelligence (AI) models and transmit an alert signal to at least one device upon prediction of the occurrence of the seizure in the subject.

Various objects, features, aspects, and advantages of the inventive subject matter will become more apparent from the following detailed description of preferred embodiments, along with the accompanying drawing figures in which like numerals represent components.

### BRIEF DESCRIPTION OF THE DRAWING FIGURES

FIG. 1A illustrates an exemplary network architecture implementing a proposed system to detect a seizure in a subject, in accordance with an embodiment of the present disclosure.

FIG. 1B illustrates an exemplary block diagram of a proposed system to detect the seizure in a subject, in accordance with an embodiment of the present disclosure.

FIG. 2A illustrates a schematic representation of interictal epochs, in accordance with an embodiment of the present disclosure.

FIG. 2B illustrates a schematic representation of preictal epochs, in accordance with an embodiment of the present disclosure.

FIG. 3A-3F illustrates a schematic representation of the ECG signal and the ECG signal after applying filtering techniques, in accordance with an embodiment of the present disclosure.

FIG. 4A illustrates an exemplary flow chart of spectral whitening (SW) filtering technique applied to the ECG signal prior extraction of one or more variations, in accordance with an embodiment of the present disclosure.

FIG. 4B illustrates an exemplary flow chart of cascaded SW filtering technique applied to the ECG signal prior to extraction of the one or more variations, in accordance with an embodiment of the present disclosure.

FIG. 5 illustrates a schematic representation of a Convolution Neural Network (CNN) architectural model for predicting an occurrence of the seizure, in accordance with an embodiment of the present disclosure.

FIG. 6A illustrates an exemplary view of an electrocardiogram (ECG) signal during an interictal stage, in accordance with an embodiment of the present disclosure.

FIG. 6B illustrates an exemplary view of the ECG signal during a preictal stage, in accordance with an embodiment of the present disclosure.

FIG. 6C illustrates an exemplary view of the ECG signal during an ictal stage, in accordance with an embodiment of the present disclosure.

FIG. 7 illustrates a schematic representation of a five-fold cross validation, in accordance with an embodiment of the present disclosure.

FIG. 8 illustrates an exemplary flow diagram of a method for detecting the seizure in the subject, in accordance with an embodiment of the present disclosure.

FIG. 9 illustrates a block diagram of an example computer system in which or with which embodiments of the present disclosure may be implemented.

### DETAILED DESCRIPTION OF THE INVENTION

The following is a detailed description of embodiments of the disclosure depicted in the accompanying drawings. The embodiments are in such details as to clearly communicate the disclosure. However, the amount of detail offered is not intended to limit the anticipated variations of embodiments; on the contrary, the intention is to cover all modifications, equivalents, and alternatives falling within the scope of the present disclosures as defined by the appended claims.

Embodiments explained herein relate to health monitoring systems, and more particularly relates to a system and method for detecting a seizure in a subject using electrocardiogram (ECG) signals, thereby enabling early seizure prediction and timely intervention before onset of the seizure. Additionally, the system and the method utilize Artificial Intelligence (AI) models to extract and classify seizure-related characteristics from the ECG signals, allowing for proactive seizure management and potentially reducing the risk of seizure-related complications.

An embodiment of the present disclosure relates to a method and a system for detecting a seizure in a subject. The method may include receiving, by one or more processors associated with a system, one or more electrocardiogram (ECG) signals from one or more sensors associated with the system. The method may include identifying, by the one or more processors, one or more patterns associated with the one or more ECG signals and extracting, by the one or more processors, one or more variations from the one or more patterns, upon identification of the one or more patterns. The method may include predicting, by the one or more processors, an occurrence of the seizure in the subject within a predefined duration based on the extracted one or more variations using Artificial Intelligence (AI) models configured within the one or more processors. The method may include transmitting, by the one or more processors, an alert signal to at least one device upon prediction of the occurrence of the seizure in the subject.

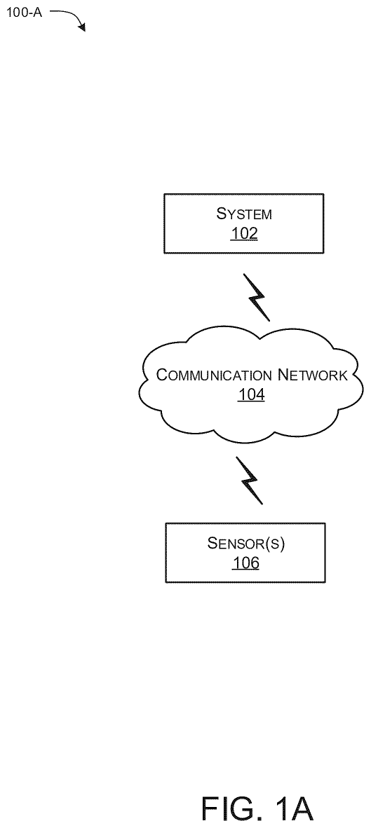

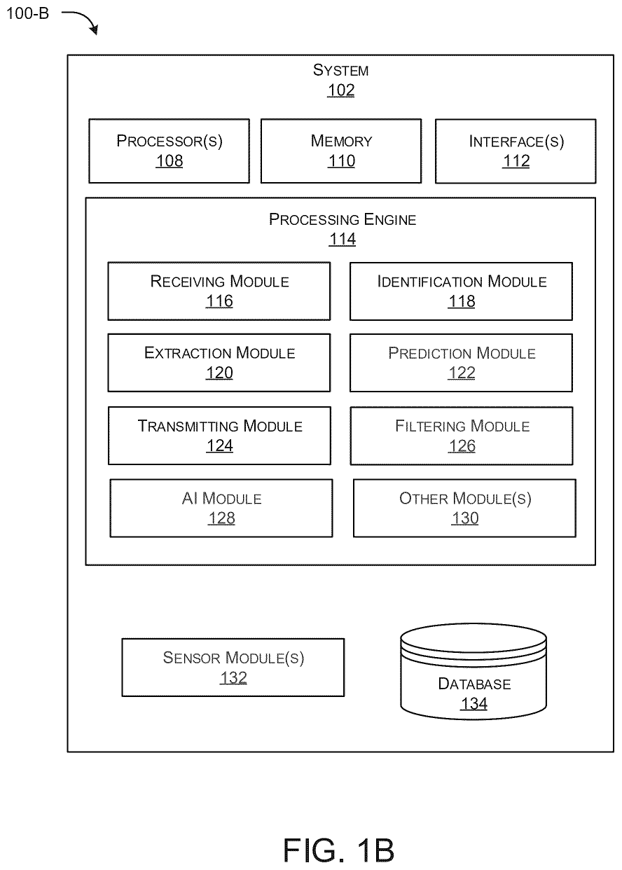

Various embodiments of the present disclosure will be explained in detail with reference to FIGS. 1A and 1B.

FIGS. 1A and 1B illustrate an exemplary network architecture 100-A and an exemplary block diagram 100-B of a proposed system 102 to detect a seizure in a subject, in accordance with an embodiment of the present disclosure.

Referring to FIGS. 1A and 1B, the system 102 to detect the seizure in the subject may include one or more processors 108, a memory 110, and an interface(s) 112. The one or more processors 108 may be implemented as one or more microprocessors, microcomputers, microcontrollers, digital signal processors, central processing units, logic circuitries, and/or any devices that manipulate data based on operational instructions. Among other capabilities, the one or more processor(s) 104 may be configured to fetch and execute computer-readable instructions stored in the memory 110 of the system 102. The memory 110 may store one or more computer-readable instructions or routines, which may be fetched and executed to predict damage of a virtual vehicle. The memory 110 may include any non-transitory storage device including, for example, volatile memory such as Random-Access Memory (RAM), or non-volatile memory such as Erasable Programmable Read-Only Memory (EPROM), flash memory, and the like.

In an embodiment, the system 102 may be communicatively connected to sensors 106 via a communication network 104. The communication network 104 may be wired communication means, or wireless communication means, or a combination thereof. In some embodiments, the wired communication means may include, but not limited to, wires, cables, data buses, optical fibre cables, and the like. In some embodiments, the wireless communication means may include, but not be limited to, telecommunication networks, Near Field Communication (NFC), Bluetooth, and the like. In some embodiments, the form factor of the data transmitted through the communication means may be any one or combination of including, but not limited to, analogue signals, electrical signals, digital signals, radio signals, infrared signals, data packets, and the like. The communication network 104 may include, by way of example but not limitation, at least a portion of one or more networks having one or more nodes, that transmit, receive, forward, generate, buffer, store, route, switch, process, or a combination thereof, etc. one or more messages, packets, signals, waves, voltage or current levels, some combination thereof, or so forth. The communication network 104 may include, by way of example but not limitation, one or more of: a wireless network, a wired network, an internet, an intranet, a public network, a private network, a packet-switched network, a circuit-switched network, an ad hoc network, an infrastructure network, a public-switched telephone network (PSTN), a cable network, a cellular network, a satellite network, a fiber optic network, or some combination thereof.

In an embodiment, the interface(s) 112 may comprise a variety of interfaces, for example, a variety of interfaces, for example, interfaces for data input and output devices, referred to as I/O devices, storage devices, and the like. The interface(s) 112 may facilitate communication of the system 102 with various devices coupled to it. The interface(s) 112 may also provide a communication pathway for one or more components of the system 102. Examples of such components include but are not limited to, processing engine(s) 114, sensor module(s) 132, and a database 134. The database 134 may include data that is either stored or generated as a result of functionalities implemented by any of the components of the processing engine(s) 114.

In an embodiment, the processing engine(s) 114 may be implemented as a combination of hardware and programming (for example, programmable instructions) to implement one or more functionalities of the processing engine(s) 110. In examples described herein, such combinations of hardware and programming may be implemented in several different ways. For example, the programming for the processing engine(s) 114 may be processor executable instructions stored on a non-transitory machine-readable storage medium and the hardware for the one or more processor(s) 104 may comprise a processing resource (for example, one or more processors 108), to execute such instructions. In the present examples, the machine-readable storage medium may store instructions that, when executed by the processing resource, implement the processing engine(s) 114. In such examples, the system 102 may comprise the machine-readable storage medium storing the instructions and the processing resource to execute the instructions, or the machine-readable storage medium may be separate but accessible to the system 102 and the processing resource. In other examples, the processing engine(s) 114 may be implemented by an electronic circuitry.

Further, the processing engine(s) 114 may include a receiving module 116, an identification module 118, an extraction module 120, a prediction module 122, a transmitting module 124, a filtering module 126, an AI module 128 and other module(s) 130. The other module(s) 130 may implement functionalities that supplement applications/functions performed by the processing engine(s) 114.

In an embodiment, the AI module 128 may be configured with an Artificial Intelligence (AI) model. The AI model may be trained and tested using a structured train-test split from various databases, ensuring robust generalization across diverse subjects and seizure types. The training phase may involve feature extraction from preprocessed ECG signals, and capturing patterns relevant to seizure prediction. The AI model may utilize Convolution Neural Network (CNN), Long Short-Term Memory (LSTM) networks, or hybrid architectures and may be trained using supervised learning on labeled datasets, where interictal, preictal, ictal, and postictal states are clearly annotated. The AI model may undergo cross-validation using separate train and test partitions defined within the datasets, ensuring it does not memorize subject-specific patterns but instead generalizes to unseen subjects. Performance metrics such as accuracy, sensitivity, specificity, and Area Under the Receiver Operating Characteristic (AUROC) curve may be monitored to optimize classification thresholds. The final model may be validated using external datasets to assess real-world applicability, ensuring high seizure prediction accuracy in subject-independent scenarios, making it suitable for clinical deployment.

In an embodiment, the receiving module 116 may be configured to receive electrocardiogram (ECG) signals from one or more sensors 106 (collectively referred to as sensors 106, hereinafter) associated with the system 102. In an embodiment, the one or more sensors 106 may include, but not limited to, ECG sensors. In an embodiment, the ECG signals may be received using the sensor module(s) 132 associated with the one or more sensors 106. The sensor module(s) 132 may capture the ECG signals and transmit the ECG signals to the receiving module 116 for further processing. The sensors 106 may be positioned on the subject for collecting the ECG signals. Further, the ECG signals may contain information related to the organs such as heart, lungs, brain and the like along with information related to the seizure. In an example scenario, the subject may be located in various settings, such as, but not limited to, a hospital, an ambulance, clinical settings, and the like. Further, the sensors 106 may be configured in wearable devices such as a sensor band, a chest band, a smartwatch, and the like.

In an embodiment, the processor 108, via the filtering module 126, may be configured to filter noise from the received ECG signals. The noise may be for example, motion artifacts, baseline wanders, powerline interference and the like. For instance, the motion artifacts may occur while wearing the sensors 106, baseline wanders may correspond to change in position of the subject and the powerline interference may be caused due to electrical noise from devices in proximity of the sensors 106. The noise may be filtered using filtering techniques, such as, notch filtering, adaptive filtering and the like.

In an embodiment, the identification module 118 may be configured to identify patterns associated with the ECG signals. The patterns may include any one or a combination of heart rate acceleration, cardiac rhythm, amplitude of the ECG signals and frequency of the ECG signals. The processor 108 may identify the patterns to assess for regular and irregular patterns that may be indicative of the seizure. For example, a sudden increase in heart rate acceleration may indicate a potential seizure or change in frequency bands of the ECG signals may indicate an onset of the seizure.

In an embodiment, the processor 108 may segment the ECG signals into predetermined time frames for identifying the patterns. To detect seizure-related trends, the ECG signals may be segmented into fixed time frames of 30 seconds, 60 seconds, 120 seconds and the like. From experimentation, the predetermined time frame of 60 seconds may be utilized for identifying the patterns, enabling an accurate seizure prediction performance.

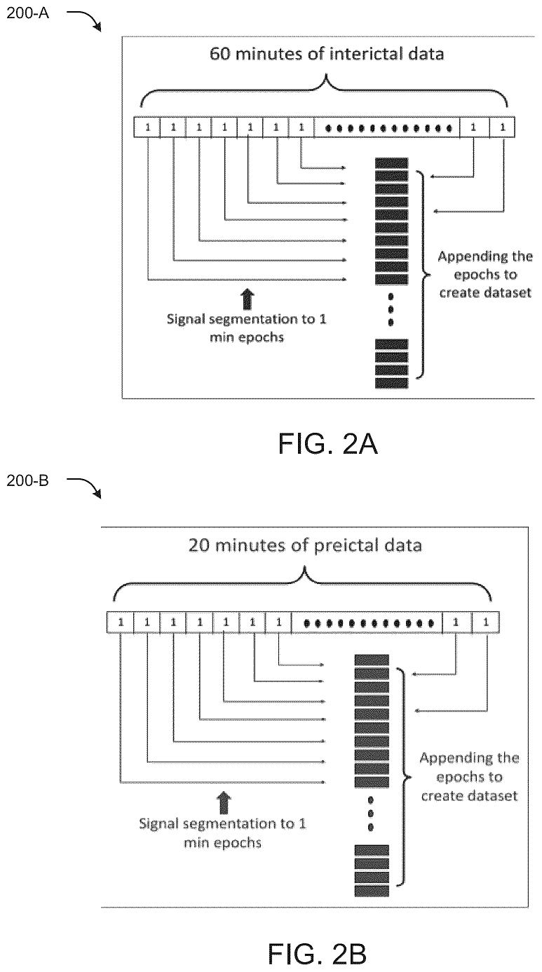

FIGS. 2A-2B illustrate a schematic representation of interictal 200-A and preictal 200-B epochs respectively, in accordance with an embodiment of the present disclosure.

In an exemplary embodiment, two different types of ECG data-interictal (non-seizure) and preictal (pre-seizure) may be divided into 1-minute epochs to create a structured dataset. Sixty minutes of interictal data may be collected from a stable period with no seizures and segmented into 1-minute epochs, each representing a non-seizure state. The epochs may be appended to form the interictal dataset, used as a baseline for comparison. Twenty minutes of preictal data may be collected before the onset of the seizure and segmented into 1-minute epochs. The epochs may be appended to form the preictal dataset, which may identify patterns leading up to the seizure. The segmentation process may allow the AI model, configured in the AI module 128, to learn features distinguishing seizure-prone (preictal) conditions from normal (interictal) conditions, improving seizure prediction accuracy.

In an exemplary scenario, the processor 108 may receive interictal ECG signals from the sensors 106. The interictal ECG signals may correspond to normal, seizure-free signals and may be used as a baseline for detecting deviations leading to seizures. Further, the processor 108 may determine the parameters associated with the interictal ECG signals. In an embodiment, the parameters may include any one or a combination of regular heartbeat patterns and non-seizure neural activity. The sensors 106 attached to the subject may capture the information corresponding to seizure activities as well as non-seizure activities. Accordingly, the processor 108 may remove the parameters (associated with non-seizure activities) from the patterns using filtering techniques. The filtering techniques may include a single stage spectral whitening (SW) and a cascaded multi-stage SW.

In an embodiment, an interictal ECG signals of 60 seconds may be obtained from the subject to receive the non-seizure related information of the subject. Upon receiving the new ECG signals from the subject, the processor 108 may detect the interictal ECG signals that contain the non-seizure related information. The single stage SW may remove or whiten the non-seizure related information may be removed to obtain seizure-related information of the subject. Further, the cascaded multi-stage SW may be applied to improve the accuracy of the SW filter. Higher-order filter coefficients are sensitive to the quantization noise of the SW coefficients and a cascaded SW filter implementation can help enhance the accuracy.

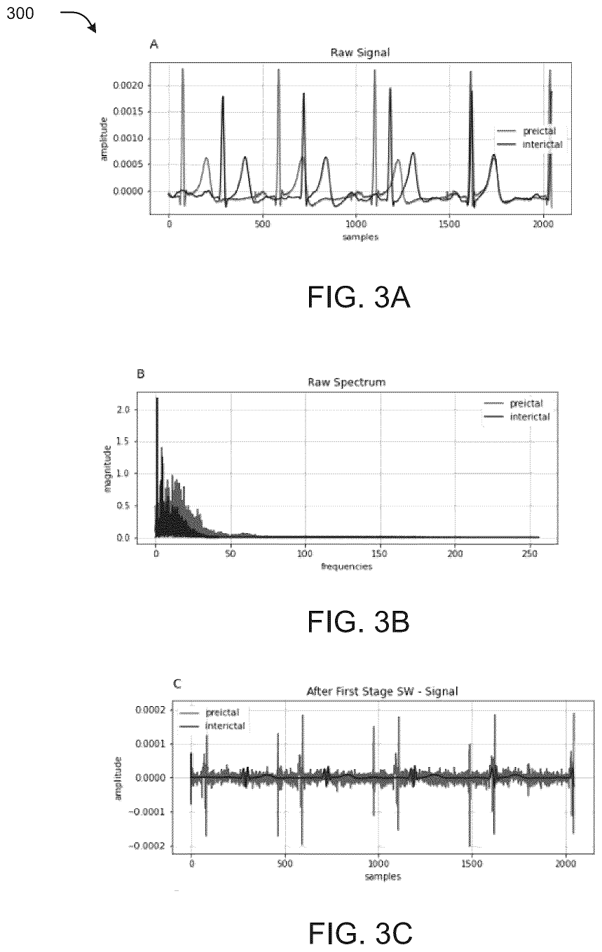

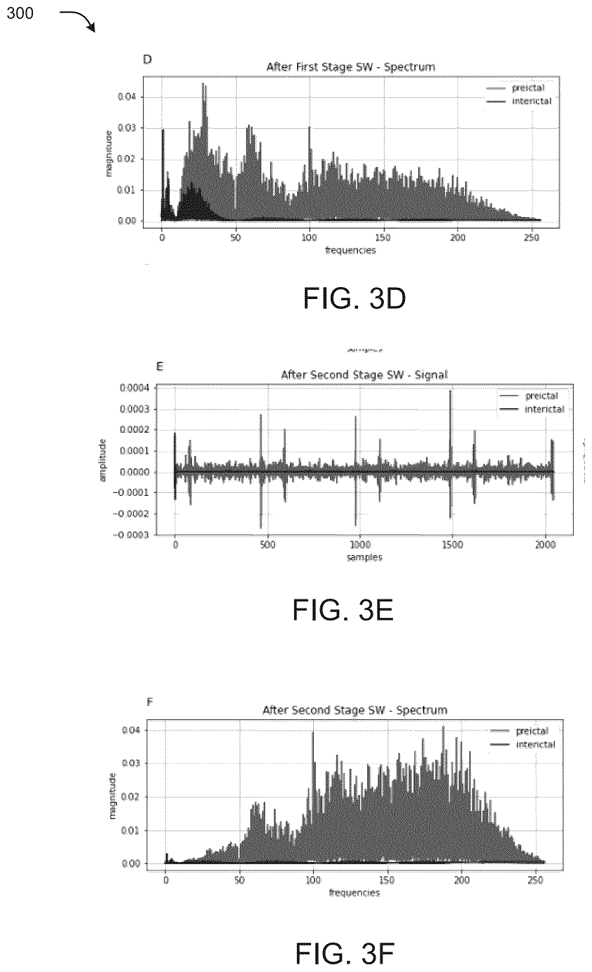

FIG. 3A-3F illustrates a schematic representation 300 of the ECG signal and the ECG signal after applying the filtering techniques, in accordance with an embodiment of the present disclosure.

In an exemplary embodiment, the filtering module 126 may apply filtering techniques prior to extracting the variations. The ECG signal may undergo an initial segmentation and processing step using the single-stage SW technique. The time-domain signal may show a smoother version of the ECG, while the frequency-domain representation highlights any major spectral changes. The cascaded multi-stage SW filtering technique may be applied for further refinement. The resulting time-domain signal may appear more filtered, with enhanced features relevant to seizure prediction. The frequency-domain spectrum may show a clearer distinction of dominant frequencies, aiding in feature extraction. The stepwise processing improves noise reduction and feature enhancement, making the extracted ECG variations more reliable for AI-based seizure detection.

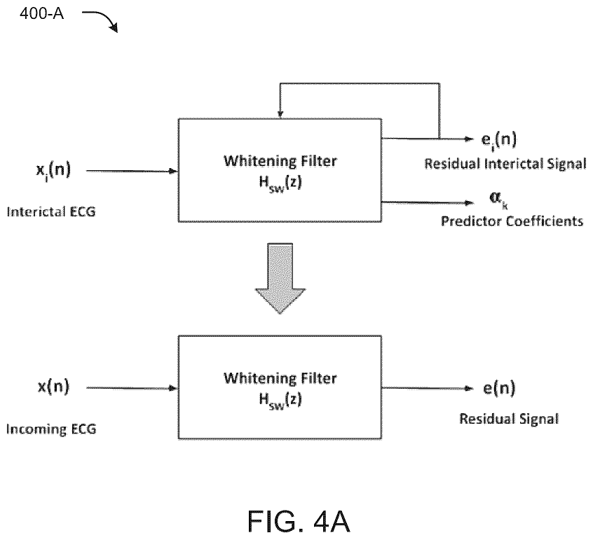

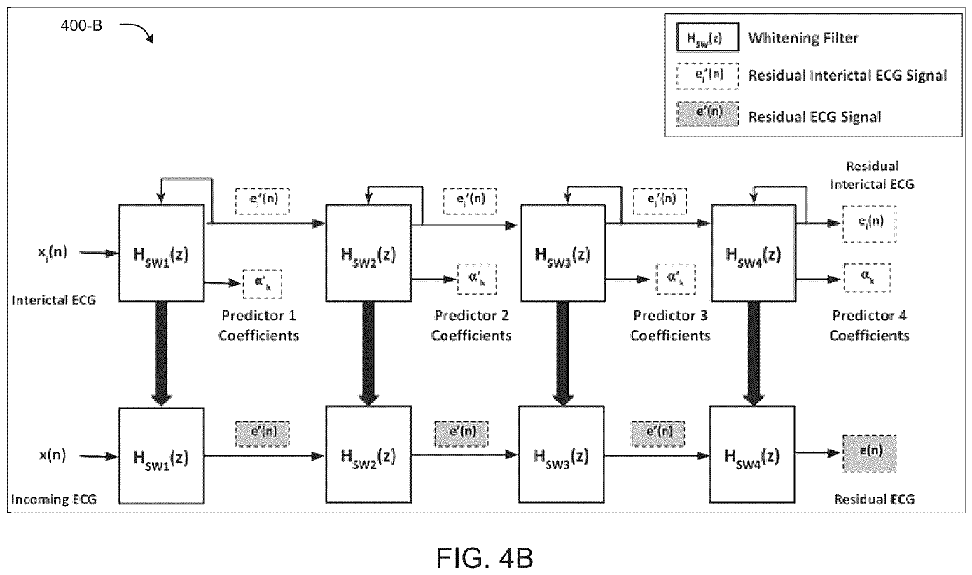

FIGS. 4A-4B illustrate an exemplary flow chart of spectral whitening (SW) filtering 400-A and cascaded SW filtering 400-B applied to the ECG signal, respectively, in accordance with an embodiment of the present disclosure.

Referring to FIG. 4A, the interictal ECG signal x[n] may be input to the SW filter H_{SW}(z). The SW filter H_{SW}(z) estimates coefficients αₖ such that the whitened spectrum of the interictal signal eᵢ(n) is flat. The residual signal obtained after the SW filtering is called e[n] which may be obtained by the given equation:

$$e [ n ] = x [ n ] - {Σ}_{k = 1}^{p} α [ k ] x [ n - k ]$$

where α[k] is a vector of SW coefficients.
α[k] can be obtained using closed-form equations or solved as an optimization problem. The αₖ coefficients may be estimated for every individual subject during respective interictal periods, to extract the non-seizure related information and subtract from the ECG signal, thereby achieving a residual signal enriched with seizure-specific information.

Referring to FIG. 4B, a second-order cascaded multi-stage SW filter H_sw1 (z) may be used to whiten the ECG signals at a first stage. The residual signals e′[n] obtained may be input to another second-order filter H_sw2 (z) to obtain the residual signal, e′[n] and process may be repeated for H_sw3 (z) and H_sw4 (z) to obtain a final residual signal e[n]. The resulting transfer function may be the product of the transfer functions of four single-stage filters given as:

H_sw(z)=H_sw1(z)·H_sw2(z)·H_sw3(z)·H_sw4(z).

The final residual signal e[n] may be utilized for evaluating the performance of the cascaded SW.

In an embodiment, the processor 108, via the filtering module 126, may determine attributes associated with the ECG signals. The attributes may include any one or a combination of patient stress levels, sleep status, and medication adherence. The attributes may interfere with the ECG signals and the processor 108 may not be able to accurately predict the occurrence of the seizure. Further, the processor 108 may remove the attributes from the patterns. The processor 108 may identify the attributes that influence the ECG signals and may remove the attributes to improve seizure prediction accuracy. For example, if the subject is stressed, the heart rate variation may drop resembling a seizure pattern. The processor 108 may adjust the changes dynamically to prevent false alerts.

In an embodiment, the extraction module 120 may be configured to extract variations from the patterns upon identification of the patterns. The variations may include heart rate variability (HRV), irregularities in the cardiac rhythm, changes in neural activity, cardiac cycle intervals, power spectral density of the frequency of the ECG signals, and waveform complexity. In an exemplary embodiment, a drop in the HRV may indicate seizure onset or skipped heartbeats with inconsistent cardiac cycle intervals may suggest autonomic instability.

In an embodiment, the processor 108, via the extraction module 120, may determine a value of the variations. Further, the processor 108 may compare the value with pre-stored value of the variations in the database 134. Further, the processor 108 may determine that the value exceeds a predefined range based on the comparison and extract the variations that exceed the predefined range. In an embodiment, the processor 108 may detect specific variations or features from the identified ECG signal patterns and determine the value of the variations. The value may be compared with pre-stored value of the variations, stored in the database 134. The stored variations may act as a reference dataset containing known seizure-related ECG characteristics collected from previous occurrences in the same subject. In an exemplary embodiment, the processor 108 may detect variations such as a sudden drop in HRV and an increase in low-frequency power in the ECG signals and determine and assign the value to the variations. The value of the detected variations may be compared against historical data stored in the database 134. If the value exceeds the predefined range and the variations match interictal ECG patterns, the processor 108 may dismiss the feature as a non-seizure feature. If a matching pattern has previously been associated with a preictal phase, the processor 108 may associate with the seizure prediction.

In an embodiment, the prediction module 122 may be configured to predict an occurrence of the seizure in the subject within a predefined duration based on the extracted one or more variations using Artificial Intelligence (AI) models configured within the AI module 128 of system 102. The system 102 may predict an impending seizure before an actual occurrence of the seizure, rather than detecting the seizure during or after onset. Upon extraction of relevant seizure-related variations, the AI module 128 may analyze the variations using the Artificial Intelligence (AI) models. The processor 108 may estimate the occurrence of the seizure within the predefined duration of 5 minutes to 20 minutes before onset of the seizure. Further, the Convolutional Neural Networks (CNNs) may be used to detect spatial patterns in ECG signals. Recurrent Neural Networks (RNNs), Long Short-Term Memory (LSTM) models may be used to identify time-dependent changes in cardiac activity. Further, hybrid AI Models (CNN+LSTM) may be used to learn both spatial and sequential dependencies for improved accuracy.

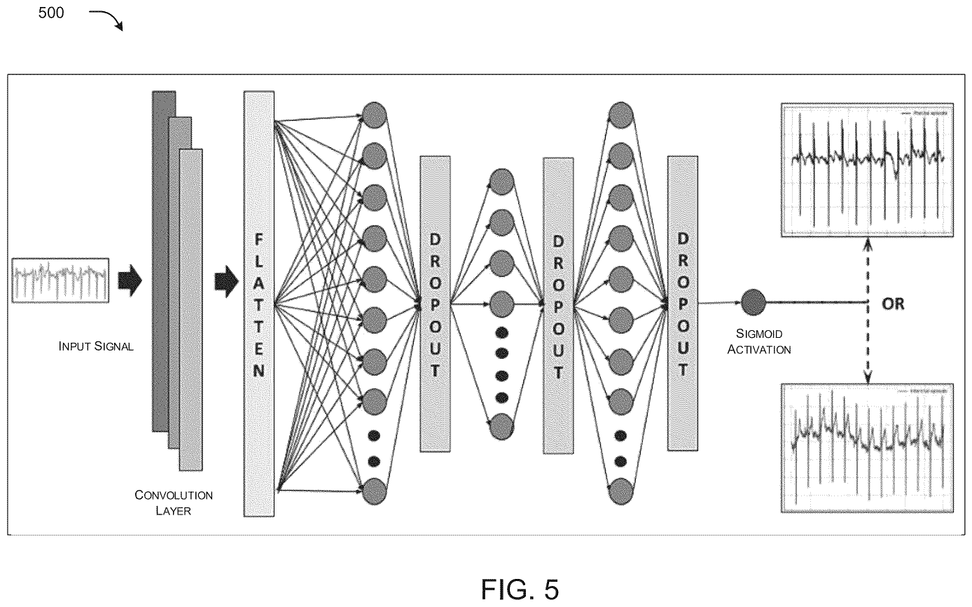

FIG. 5 illustrates a schematic representation of a Convolution Neural Network (CNN) architectural model 500 configured in the AI module 128 for predicting the occurrence of the seizure, in accordance with an embodiment of the present disclosure.

Referring to FIG. 5, the CNN may be used to classify the SW interictal and preictal signals of the ECG signal to evaluate the seizure prediction performance. The CNN may include two convolutional layers with a rectified linear unit (ReLU) activation. Further, a flattening layer and two dense layers with a ReLU activation and 0.5 dropout for regularization may follow the convolution layers. The Adam optimizer with a learning rate of 0.0001 may be used and the output may be predicted using a single dense node with sigmoid activation. It may be appreciated that the implementation of the disclosed embodiments may not be restricted to the configuration described in FIG. 5 and other possible implementations may also be envisaged.

In an embodiment, the processor 108, via the prediction module 122, may determine stages associated with the seizure based on the extracted variations. The stages may include preictal stage, ictal stage, interictal stage, and postictal stage. Further, the processor 108 may classify the variations into the stages using the AI models upon extraction of the variations. The processor 108 determines which stage of seizure progression the patient is currently experiencing, and the seizures may evolve through the stages.

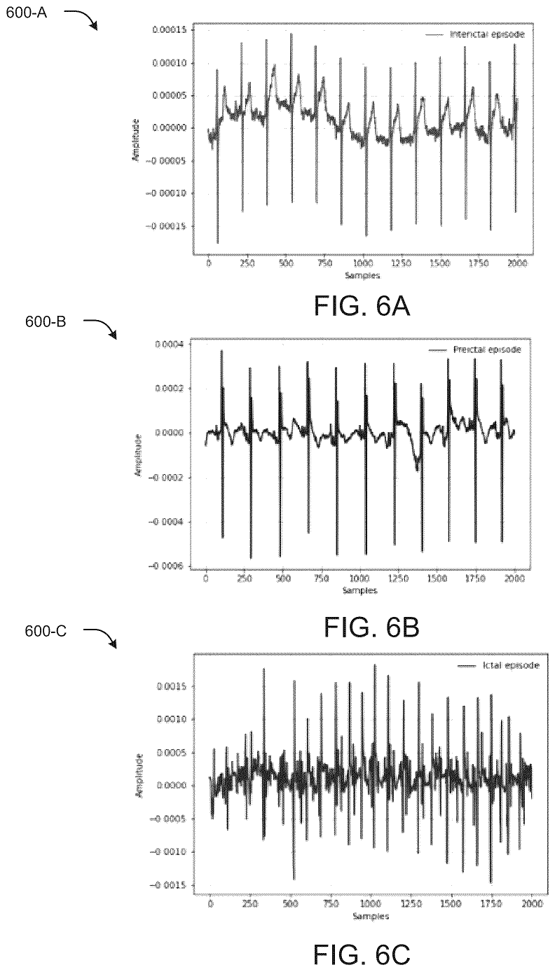

FIGS. 6A-6C illustrate an exemplary view of an electrocardiogram (ECG) signal during an interictal stage 600-A, a preictal stage 600-B, and an ictal stage 600-C, respectively, in accordance with an embodiment of the present disclosure.

Referring to FIGS. 6A to 6C, the preictal stage may be the period before seizure onset and may be characterized by increased autonomic instability, HRV reduction, and spectral energy shifts. The processor 108 may provide early warnings at the preictal stage to allow patient intervention. Further, the interictal stage may be a non-seizure state, where the variations may match baseline the ECG signals. Further, the ictal stage may be an active seizure period with sudden high-amplitude ECG variations, HRV collapse, and disordered spectral patterns. Further, the postictal stage may be a recovery phase after the seizure.

In an embodiment, the prediction module 122 may assign a score to the variations upon classification of the variations into the stages. The processor 108 may quantify seizure risk by assigning the score based on the extracted variations and the classification. The score may differentiate true seizures from false alarms and may optimize prediction confidence. The score may be assigned based on degree of similarity between real-time ECG variations and historical seizure patterns stored in the database 134. For example, high-impact variations such as a drop in the HRV may be assigned a higher score. Further, the processor 108 may determine that the score exceeds a predefined threshold. Further, the processor 108 may detect the occurrence of the seizure in the subject within the predefined duration, upon determination that the score exceeds the predefined threshold. If the score exceeds the threshold, the processor 108 may confirm the occurrence of the seizure. In an exemplary embodiment, if the predetermined threshold is 0.85 for seizure detection and the processor 108 calculates the score as 0.92, it may indicate a high likelihood of seizure onset and the processor 108 may confirm the occurrence of the seizure.

In an embodiment, the processor 108 may validate the occurrence of the seizure by comparing the predicted seizure with historical seizure data stored in the database 134. The processor 108 may cross-check the detected variations against previous seizures logged in the historical seizure data.

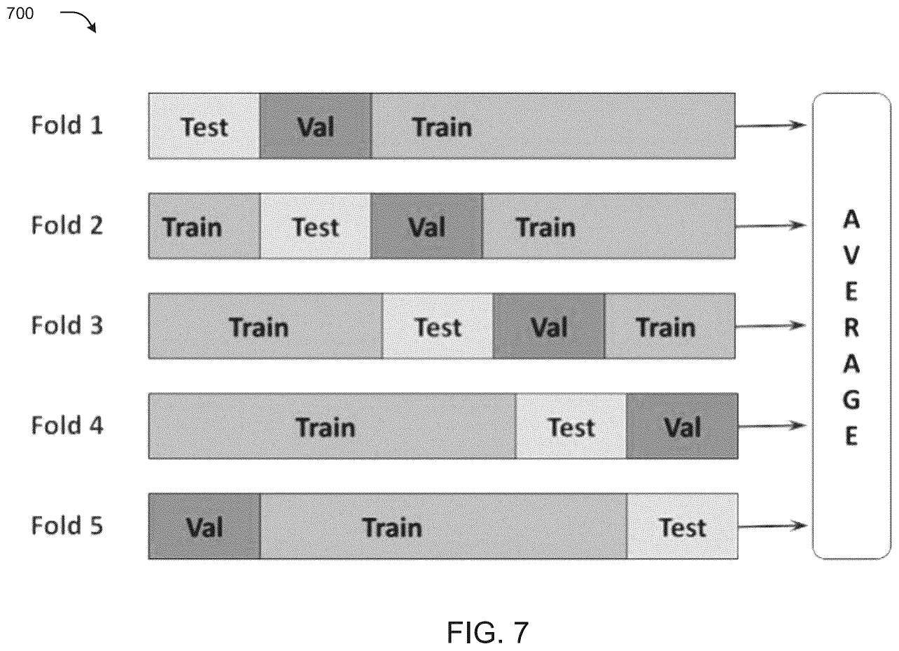

FIG. 7 illustrates a schematic representation of a five-fold cross validation, in accordance with an embodiment of the present disclosure.

In an exemplary embodiment, referring to FIG. 7, a five-fold cross-validation method may be utilized and each dataset may be divided into 5 equal-sized subsets, chronologically according to subjects. The sample size of each fold may vary depending on the selection of the subjects. The CNN model may undergo training using three of the subsets and may be tested and validated on the remaining subsets respectively. Predictions from the test subsets may be recorded, and repeated five times, with a different subset serving as the test data in each iteration.

In an embodiment, the transmitting module 124 may be configured to transmit an alert signal to at least one device upon prediction of the occurrence of the seizure in the subject. The at least one device may include, but not limited to, a mobile device, a smartwatch, a laptop, a haptic feedback device and the like. For instance, the processor 108 may detect a high seizure probability score of 0.95. The alert signal may be sent to caregivers with real-time GPS location tracking. In another instance, the smartwatch may vibrate to notify the subject of the seizure risk and if the subject does not respond, the processor 108 may automatically call emergency services.

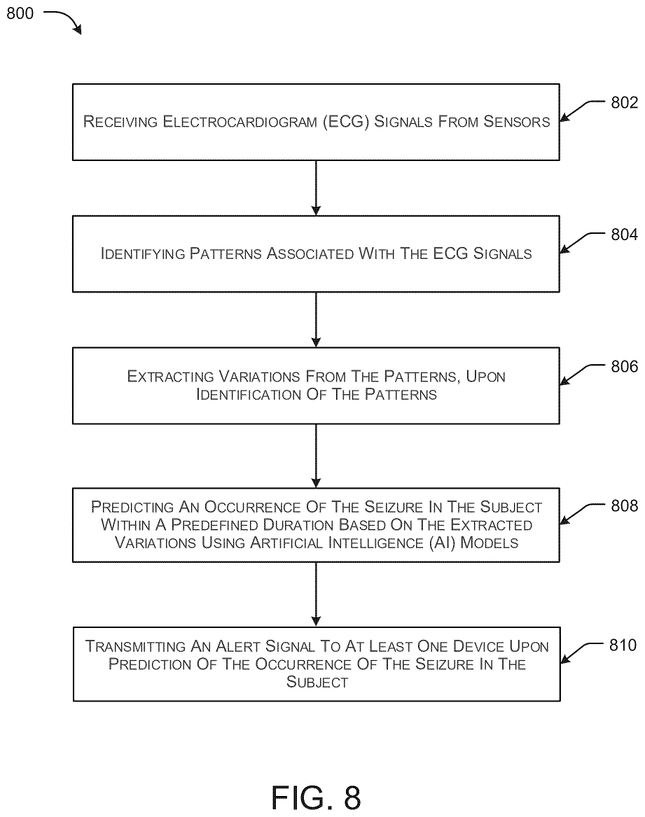

FIG. 8 illustrates a flow diagram illustrating an exemplary method 800 for detecting the seizure in the subject, in accordance with an embodiment of the present disclosure.

Referring to FIG. 8, at block 802, the method 800 may include receiving, by the processors 108 associated with the system 102, the electrocardiogram (ECG) signals from the sensors 106 associated with the system 102

At block 804, the method 800 may include identifying, by the processors 108, the patterns associated with the ECG signals.

At block 806, the method 800 may include extracting, by the processors 108, the variations from the patterns, upon identification of the patterns.

At block 808, the method 800 may include predicting, by the processors 108, the occurrence of the seizure in the subject within the predefined duration based on the extracted variations using Artificial Intelligence (AI) models configured within the system 102.

At block 810, the method 800 may include transmitting, by the processors 108, the alert signal to the at least one device upon prediction of the occurrence of the seizure in the subject.

Thus, the present disclosure proposes a system (e.g., 102 as represented in FIGS. 1A and 1B) and a method (e.g., 800 as represented in FIG. 8) for detecting a seizure in a subject. By incorporating ECG processing techniques and AI-based predictive models, the system 102 and the method 800 aim to provide a robust, effective, and flexible solution for early seizure prediction and real-time monitoring of seizures.

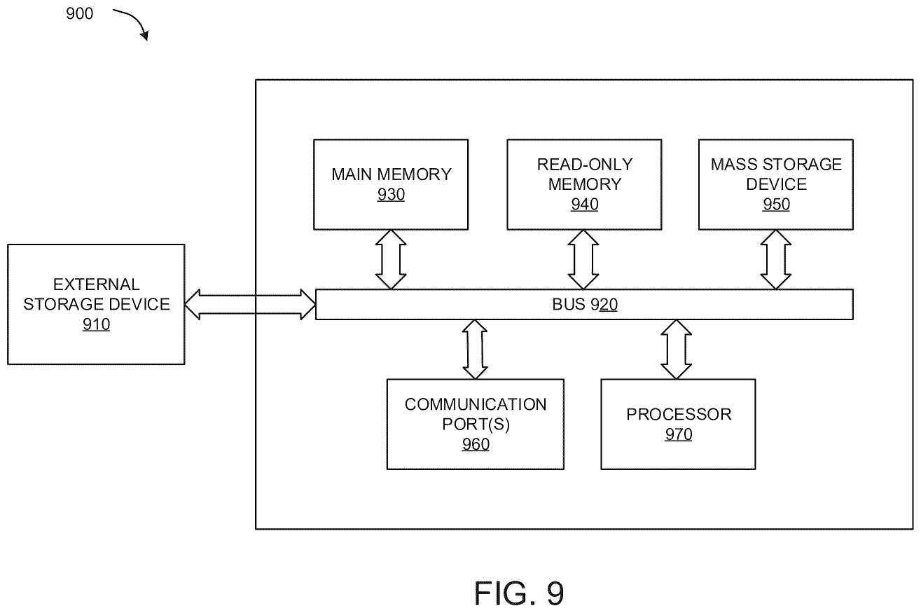

FIG. 9 illustrates a block diagram of an example computer system 900 in which or with which embodiments of the present disclosure may be implemented.

As shown in FIG. 9, the computer system 900 may include an external storage device 910, a bus 920, a main memory 930, a read-only memory 940, a mass storage device 950, communication port(s) 960, and a processor 970. A person skilled in the art will appreciate that the computer system 900 may include more than one processor and communication ports. The processor 970 may include various modules associated with embodiments of the present disclosure. The communication port(s) 960 may be any of an RS-232 port for use with a modem-based dialup connection, a 10/100 Ethernet port, a Gigabit or 10 Gigabit port using copper or fibre, a serial port, a parallel port, or other existing or future ports. The communication port(s) 960 may be chosen depending on a network, such a Local Area Network (LAN), Wide Area Network (WAN), or any network to which the computer system 900 connects. The main memory 930 may be random access memory (RAM), or any other dynamic storage device commonly known in the art. The read-only memory 940 may be any static storage device(s) including, but not limited to, a Programmable Read Only Memory (PROM) chips for storing static information e.g., start-up or basic input/output system (BIOS) instructions for the processor 970. The mass storage device 950 may be any current or future mass storage solution, which may be used to store information and/or instructions.

The bus 920 communicatively couples the processor 970 with the other memory, storage, and communication blocks. The bus 920 can be, e.g., a Peripheral Component Interconnect (PCI)/PCI Extended (PCI-X) bus, Small Computer System Interface (SCSI), universal serial bus (USB), or the like, for connecting expansion cards, drives, and other subsystems as well as other buses, such a front side bus (FSB), which connects the processor 970 to the computer system 900.

Optionally, operator and administrative interfaces, e.g. a display, keyboard, and a cursor control device, may also be coupled to the bus 920 to support direct operator interaction with the computer system 900. Other operator and administrative interfaces may be provided through network connections connected through the communication port(s) 960. In no way should the aforementioned exemplary computer system 900 limit the scope of the present disclosure.

While the foregoing describes various embodiments of the present disclosure, other and further embodiments of the present disclosure may be devised without departing from the basic scope thereof. The scope of the present disclosure is determined by the claims that follow. The present disclosure is not limited to the described embodiments, versions or examples, which are included to enable a person having ordinary skill in the art to make and use the present disclosure when combined with information and knowledge available to the person having ordinary skill in the art.

### ADVANTAGES OF THE PRESENT INVENTION

The present invention enables accurate and early seizure detection by leveraging ECG signals and artificial intelligence (AI)-based predictive models, ensuring timely medical intervention.

The present invention enables real-time monitoring of cardiac and neural activity, allowing for assessment of a physiological state of a subject and detecting seizure-related abnormalities.

The present invention allows filtering and removal of non-seizure-related parameters, improving the reliability of seizure predictions.

The present invention provides efficient segmentation of ECG signals into predefined time frames, facilitating improved feature extraction.

The present invention facilitates classification of extracted variations into different ictal stages using the AI models, improving the accuracy of seizure detection.

The present invention assigns a score to detected seizure variations, ensuring that only high-probability events trigger an alert, thereby reducing false positives.

We claim:

1. A method for detecting a seizure in a subject, the method comprising:
receiving, by one or more processors associated with a system, one or more electrocardiogram (ECG) signals from one or more sensors associated with the system;
identifying, by the one or more processors, one or more patterns associated with the one or more ECG signals;
extracting, by the one or more processors, one or more variations from the one or more patterns, upon identification of the one or more patterns;
predicting, by the one or more processors, an occurrence of the seizure in the subject within a predefined duration, based on the extracted one or more variations using Artificial Intelligence (AI) models configured within the system; and
transmitting, by the one or more processors, an alert signal to at least one device upon prediction of the occurrence of the seizure in the subject.

2. The method as claimed in claim 1, wherein the one or patterns comprise any one or a combination of: heart rate acceleration, cardiac rhythm, amplitude of the one or more ECG signals, and frequency of the one or more ECG signals.

3. The method as claimed in claim 1, wherein the one or more variations comprise any one or a combination of: heart rate variability, irregularities in cardiac rhythm, changes in neural activity, changes in cardiac cycle intervals, variation in power spectral density of a frequency of the one or more ECG signals, and waveform complexity.

4. The method as claimed in claim 1, further comprising filtering, by the one or more processors, noise from the received one or more ECG signals prior to identifying the one or more patterns.

5. The method as claimed in claim 1, wherein extracting, by the one or more processors, the one or more variations from the one or more patterns, comprises:
determining, by the one or more processors, a value of the one or more variations;
comparing, by the one or more processors, the value with pre-stored value of the one or more variations in a database associated with the system;
determining, by the one or more processors, that the value exceeds a predefined range based on the comparison; and
extracting, by the one or more processors, the one or more variations based on the determination that the value exceeds the predefined range.

6. The method as claimed in claim 1, further comprising:
receiving, by the one or more processors, one or more interictal ECG signals from the one or more sensors;
determining, by the one or more processors, one or more parameters associated with the one or more interictal ECG signals; and
removing, by the one or more processors, the one or more parameters from the one or more patterns using one or more filtering techniques prior to extracting the one or more variations.

7. The method as claimed in claim 6, wherein the one or more parameters comprise any one or a combination of: regular heartbeat patterns and non-seizure neural activity.

8. The method as claimed in claim 1, further comprising:
determining, by the one or more processors, one or more attributes associated with the one or more ECG signals, wherein the one or more attributes comprise any one or a combination of: patient stress levels, sleep status, and medication adherence; and
removing, by the one or more processors, the one or more attributes from the one or more patterns prior to extracting the one or more features.

9. The method as claimed in claim 1, wherein predicting, by the one or more processors, the occurrence of the seizure in the subject within the predefined duration based on the extracted one or more variations using the AI models, comprises:
determining, by the one or more processors, one or more stages associated with the seizure based on the extracted one or more variations;
classifying, by the one or more processors, the one or more variations into the one or more stages using the AI models upon extraction of the one or more variations to predict the occurrence of the seizure in the subject;
assigning, by the one or more processors, a score to the one or more variations upon classification of the one or more variations into the one or more stages;
determining, by the one or more processors, that the score exceeds a predefined threshold; and
detecting, by the one or more processors, the occurrence of the seizure in the subject within the predefined duration, upon determination that the score exceeds the predefined threshold.

10. The method as claimed in claim 9, wherein the one or more stages comprise any one of: preictal stage, ictal stage, interictal stage, and postictal stage.

11. A system for detecting a seizure in a subject, the system comprising:
one or more processors; and
a memory operatively coupled with the one or more processors, wherein the memory comprises one or more instructions which, when executed, cause the one or more processors to:
receive one or more electrocardiogram (ECG) signals from one or more sensors associated with the system;
identify one or more patterns associated with the one or more ECG signals;
extract one or more variations from the one or more patterns, upon identification of the one or more patterns;
predict an occurrence of the seizure in the subject within a predefined duration based on the extracted one or more variations using Artificial Intelligence (AI) models; and
transmit an alert signal to at least one device upon prediction of the occurrence of the seizure in the subject.

12. The system as claimed in claim 11, wherein the one or patterns comprise any one or a combination of: heart rate acceleration, cardiac rhythm, amplitude of the one or more ECG signals, and frequency of the one or more ECG signals.

13. The system as claimed in claim 11, wherein the one or more variations comprise any one or a combination of: heart rate variability, irregularities in cardiac rhythm, changes in neural activity, changes in cardiac cycle intervals, variation in power spectral density of a frequency of the one or more ECG signals, and waveform complexity.

14. The system as claimed in claim 11, wherein the one or more processors are configured to filter noise from the received one or more ECG signals prior to the identification of the one or more patterns.

15. The system as claimed in claim 11, wherein to extract the one or more variations from the one or more patterns, the one or more processors are configured to:
determine a value of the one or more variations;
compare the value with pre-stored value of the one or more variations in a database associated with the system;
determine that the value exceeds a predefined range based on the comparison; and
extract the one or more variations based on the determination that the value exceeds the predefined range.

16. The system as claimed in claim 11, wherein the one or more processors are configured to:
receive one or more interictal ECG signals from the one or more sensors;
determine one or more parameters associated with the one or more interictal ECG signals; and
remove the one or more parameters from the one or more patterns using one or more filtering techniques prior to the extraction of the one or more variations.

17. The system as claimed in claim 16, wherein the one or more parameters comprise any one or a combination of: regular heartbeat patterns and non-seizure neural activity.

18. The system as claimed in claim 11, wherein the one or more processors are configured to:
determine one or more attributes associated with the one or more ECG signals, wherein the one or more attributes comprise any one or a combination of: patient stress levels, sleep status, and medication adherence; and
remove the one or more attributes from the one or more patterns prior to the extraction of the one or more features.

19. The system as claimed in claim 11, wherein to predict the occurrence of the seizure in the subject within the predefined duration based on the extracted one or more variations using the AI models, the one or more processors are configured to:
determine one or more stages associated with the seizure based on the extracted one or more variations;
classify the one or more variations into the one or more stages using the AI models upon extraction of the one or more variations to predict the occurrence of the seizure in the subject;
assign a score to the one or more variations upon classification of the one or more variations into the one or more stages;
determine that the score exceeds a predefined threshold; and
detect the occurrence of the seizure in the subject within the predefined duration, upon determination that the score exceeds the predefined threshold.

20. The system as claimed in claim 19, wherein the one or more stages comprise any one of: preictal stage, ictal stage, interictal stage, and postictal stage.
